(19) United States
(12) Patent Application Publication
(10) Pub. No.: US 2023/0241380 A1
(43) Pub. Date: Aug. 3, 2023

(54) System and method for increasing absorption of medications and cosmeceuticals through the skin of the user

(71) Applicants: Scott Sheftel, Tucson, AZ (US); Jeffry Skiba, Chandler, AZ (US)

(72) Inventors: Scott Sheftel, Tucson, AZ (US); Jeffry Skiba, Chandler, AZ (US)

(21) Appl. No.: 18/131,777

(22) Filed: Apr. 6, 2023

Related U.S. Application Data
(63) Continuation-in-part of application No. 17/920,177, filed on Oct. 20, 2022.
Application No. 17/920,177 is a continuation-in-part of application No. 15/823,076, filed on Nov. 27, 2017, now Pat. No. 11,124,901.
(60) Provisional application No. 63/327,968, filed on Apr. 6, 2022.

(51) Int. Cl. A61N 1/30; A61N 1/04

(52) U.S. Cl. CPC A61N 1/306; A61N 1/0496

(57) ABSTRACT

A material formed of a substrate material and particles of an oxidizable metal disbursed in a desired pattern are provided. The particles are dispersed within the substrate during formation of the substrate and exposed at least in part on the surface of the substrate to form an iontophoretic battery when contacted with the skin of a user.

### CROSS REFERENCE TO RELATED APPLICATION

This application is a continuation in-part of U.S. patent application Ser. No. 17/920,177, filed Oct. 20, 2022, which is a continuation in-part of U.S. patent application Ser. No. 15/823,076, filed Nov. 27, 2017 and issued as U.S. Pat. No. 11,124,901, the contents of all of which are incorporated herein by reference. This application also claims the benefit of Provisional App. No. 63/327,968, the contents of which is herein incorporated by reference.

### BACKGROUND OF THE INVENTION

### Field of the Invention

The present invention relates to a method and apparatus for facilitating wound care increasing absorption of substances through the skin of the user. The invention has particular utility in connection with increasing absorption of medications through the skin, and will be described in connection with such utility, although other utilities, such as increasing the speed or depth of absorption of other substances including, for example, cosmeceuticals, is contemplated.

### Description of Related Art

Skin comprises epidermis, dermis, and subcutaneous adipose tissue. When medicines are applied to the skin, the epidermis, i.e., the upper layer of the skin, ordinarily considers the medicines as foreign substances and hinders absorption of the medicine. As a result, due to the influence of molecular size, bio characteristics, biochemical phenomena, and the like, the amount of medicines practically absorbed into the skin is very small.

In order to overcome this problem, iontophoresis has been developed to increase absorption of medicines through the skin. Iontophoresis involves generating a micro current to flow through the skin thereby effectively increasing absorption of components contained in the medicines having electrical affinity with the skin, by electrical repulsive force. Iontophoresis may be used, for example, so that topical vitamin C, which is an anti-oxidant and a co-factor in skin growth, is absorbed into the skin providing cellular stimulation and anti-oxidant protection. Topical vitamin C is used for skin health and improvement of wrinkles. Other uses of iontophoresis can range from medication delivery, such as with prescription medications, to localized delivery without systemic side-effects.

Current iontophoresis apparatuses induce electric fields on and around the skin by attaching patches to the skin and allowing micro current to flow from the patches into the skin. However, current iontophoresis apparatuses require a source of electricity, e.g., a battery or connection to electrical mains, wires, circuits, etc., which are costly and bulky, and are inconvenient to use, resulting in reduced patient compliance. Thus, a need exists for a self-contained, low cost method and apparatus for delivering medicines for absorption through the skin. In addition to the iontophoretic effect on the skin, directed electric micro current can also stimulate the skin, enhance blood flow and tissue oxygenation, as well as an enhance cellular response with increased protein synthesis, amino acid transport and increased ATP (mitochondrial energy) synthesis. The net effect is localized collagen stimulation addressing wrinkles, complexion and the health of the skin.

So as to reduce the complexity and length of the Detailed Specification, and to fully establish the state of the art in certain areas of technology, Applicant(s) herein expressly incorporate(s) by reference all of the following materials identified in each numbered paragraph below. The incorporated materials are not necessarily “prior art” and Applicant(s) expressly reserve(s) the right to swear behind any of the incorporated materials.

Applicant(s) believe(s) that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), applicant(s) will amend the specification to expressly recite the essential material that is incorporated by reference as allowed by the applicable rules.

### BRIEF SUMMARY OF THE INVENTION

The above and other objects may be achieved using devices for amplified activity of a treatment substance comprising a substrate with a fixed desired pattern of particles of an oxidizable metal dispersed within the substrate in discrete electrically isolated locations. The metal particles are at least partially exposed on the surface of the substrate to interact with air and the surface on which the device is placed to function as an air-or oxygen-metal battery for ion exchange with the skin of a user. A treatment substance that may include an ointment or cream may be carried by the substrate to also contact the skin of the user to facilitate ion exchange with the skin to drive the treatment substance into the skin by iontophoresis. The treatment substance may reside in treatment reservoirs formed in a porous substrate.

The desired pattern may be designed to conform to blood circulation patterns and/or nerve or underlying muscle patterns of the human or animal and the particles may be 0.1 mm to 5 mm in diameter, preferably about 0.1 to 3 mm, more preferably 0.2 to 2 mm, even more preferably 0.3 to 1 mm, most preferably 0.4 to 0.5 mm. The metal particles may comprise a metal, metal oxide, or metal salt, and in a particular embodiment may be selected from the group consisting of elemental zinc particles, zinc oxide particles, and zinc salt particles. The metal particles may alternatively be selected from aluminum, iron, copper, magnesium, or any combination of the elemental metals, their oxide, or a salt thereof.

The pattern can cover from about 10% to 90% of the surface of the cover layer, preferably from about 15% to about 75%, more preferable from about 25% to about 50%, most preferably from about 30% to about 40%. 9. The pattern may include a pattern in which the particles are substantially evenly spaced from one another, in particular at spacings from 0.1 to 3 mm, preferably 0.2 to 2 mm, more preferably 0.3 to 1.5 mm, most preferably 0.5 to 1 mm.

The device may further including an adhesive pad or adhesive tabs adapted to fix the substrate to the skin of the user.

The above and other objects may be achieved using methods involving providing a device as described above and disposing the device in contact with the skin\ of a human or animal. The metal particles form a first half cell of a battery circuit and the body of the human or animal forms a second half cell of the battery circuit with oxygen. The substance is then driven into the skin of the human or animal by iontophoresis.

The desired pattern may be designed to conform to blood circulation patterns and/or nerve or underlying muscle patterns of the human or animal and the particles may be 0.1 mm to 5 mm in diameter, preferably about 0.1 to 1 mm, more preferably 0.1 to 0.5 mm, and even more preferably 0.2 to 0.4 mm. The metal particles may comprise a metal, metal oxide, or metal salt, and in a particular embodiment may be selected from the group consisting of elemental zinc particles, zinc oxide particles, and zinc salt particles. The metal particles may alternatively be selected from aluminum, iron, copper, magnesium, or any combination of the elemental metals, their oxide, or a salt thereof.

The pattern can cover from about 10% to 90% of the surface of the cover layer, preferably from about 15% to about 75%, more preferable from about 25% to about 50%, most preferably from about 30% to about 40%. 9. The pattern may include a pattern in which the particles are substantially evenly spaced from one another, in particular at spacings from 0.1 to 3 mm, preferably 0.2 to 2 mm, more preferably 0.3 to 1.5 mm, most preferably 0.5 to 1 mm.

The method may also involve fixing the substrate to a user using a strap, an adhesive pad, or adhesive tabs.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts a graphical representation of side view of a fibrous substrate in accordance with an embodiment of the invention.

FIG. 2 depicts a graphical representation of a non-fibrous substrate in accordance with an embodiment of the invention.

FIG. 3 depicts a magnified view of a porous substrate according to an embodiment of the invention.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

In the following description, the term “metal particles” may include elemental metal particles of metals capable of forming metal-air electrochemical cells, and oxides and salts thereof. Preferred are zinc metal particles and oxides and salts thereof, although other metals and oxides and salts thereof may be used including aluminum, iron, copper, or magnesium.

The term “fibers” may comprise both natural and synthetic fibers, filaments and threads, although synthetic fibers are preferred, in particular, fibers formed of thermoplastic or thermosetting plastic materials, and/or adhesive-coated fibers.

Zinc air batteries have been found to have several beneficial uses and applications. The standard cell potential (E°_{cell}) of the zinc-air battery is 1.65 V. The standard reduction potentials of the half-reactions of zinc-air batteries are:

ZnO(s)+H₂O(l)+2e⁻→Zn(s)+2OH⁻(aq) E°_{red}=−1.25V

O₂(g)+2H₂O(l)+4e⁻→4OH⁻(aq) E°_{red}=0.401V

Elemental zinc particles or particles of zinc salt against the skin results in secondary reactions to form zinc complexes beneficial to the host. The ability to deliver topical zinc to the surface of the skin can have beneficial effects provided the topical zinc is in the correct elemental presentation, availability and configuration.

Metal particles 16 advantageously may be used as a half cell in an oxidation/reduction reaction in contact with the skin to produce an electric field, or a plurality of electric fields that sum to a net zero voltage overall. The battery consists of an “air” electrode, an electrolyte, and a metal electrode that may be oxidized when the air electrode produces hydroxyl ions in the aqueous electrolyte. The hydroxyl ions oxidize the metal, a process that releases electrons to form an electric current. The battery may be recharged by reversing the process with oxygen being released at the air electrode.

When directed into the skin, the electric field alters skin membrane permeability, allowing substances such as pharmaceuticals to pass more easily into the skin. The electric field produced by metal particle patterns in contact with the skin provides a field direction, i.e., negative in the ion pattern and positive in the tissue. As a result, substances may be absorbed into the skin towards a positive pull to the tissue of the patient, by electrical repulsive force, i.e., negative to positive attraction, like iontophoresis. Thus, the present invention provides a self-contained external battery-free iontophoresis apparatus for driving substances such as pharmaceuticals into the skin of the wearer.

Referring to FIG. 1, a self-contained, battery-free iontophoresis apparatus or device 10 in accordance with the present invention takes the form of a flexible substrate 14 carrying a plurality of spaced lines or lines of metal particles 16. The substrate 14 may be formed of a sheet material such as an air permeable or non-air permeable textile or fabric, a fibrous mesh, non-fibrous mesh, adhesive coated textile, a flexible polymer, or other flexible substrate that may hold the particles 16 in a substantially fixed relative position.

Preferably the metal particles are zinc particles and have an average particle size of between 1 and 100 nanometers, more preferably 1 to 10 microns, and even more preferably about 5 microns. The metal particles 16 may be printed on the substrate 14, or extruded or melt spun at the time of fiber formation as taught by our aforesaid patents and pending application, or may be uniformly mixed into a liquid polymer mix that is allowed to cure. Polyethylene is a material of choice for forming the fibers for releasing zinc ions.

The amount of zinc and the surface area of the zinc or other metal used is a function of particle size and availability to create the battery. The amount of total zinc per unit volume will decide the capacity of the battery. When the zinc runs out the battery will also die out, unless there is recharging going on, e.g. due to ions present in the skin.

Preferably, but not necessarily, opposite ends of the device 10 may include adhesive or adhesive tabs shown in phantom at 22 so that the iontophoresis device may be fixed to the skin of a user. Alternatively, the iontophoresis device 10 may be held in place by a wrap or the like.

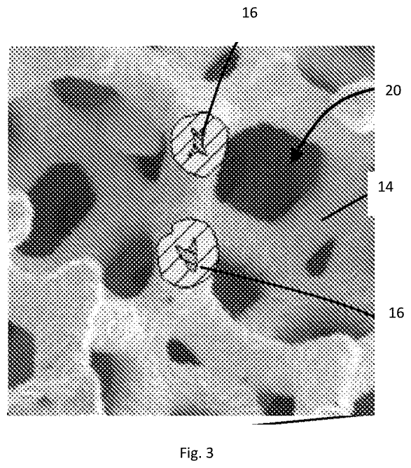

As taught in our aforesaid '761 and '172 patents or as described in our '901 patent, and as shown in FIG. 3, the metal particles are discontinuously and substantially uniformly distributed on the surface of substrate 16, in imaginary spaced lines or lines of dots, across the surface area of the substrate 16, at least in part.

The size and distance between particle reservoirs 16 makes a difference on the electric fields that created by the batter device 10. The ability to generate a field from a group of charged particles is necessary to create electric currents and field related effects such as ROS upregulation. In addition, the amount of metal in the reservoir determines the charge capacity—i.e., the more metal present, the longer the battery life.

In the case of cellular stimulation, the field shape needs to be small to reduce local current flow. Cells relate to small electric fields, not one big field which would be created by large electrodes, and physiologic fields do not exist in large electrodes. The use of substantially fixed relative patterns allows the placement of metal Zn to create these mini-electric fields. The printed patterns may be created through the thread count of fibers in a fabric or the dispersal of particles in polymer gel or other applicator that can hold the particles in a specific spacial configuration. Typically, the lines or lines of dots are substantially evenly spaced at spacings from 0.1 to 3 mm, preferably 0.2 to 2 mm, more preferably 0.3 to 1.5 mm, most preferably 0.5 to 1.0 mm.

Each particle 16 acts as a potentiator for a charge and particles separated by very small distances will create higher charge densities at the particles 16 than between the particles 16. A layer may be formed including a plurality of particles on the outer surface of the device 10. The particle reservoirs 16 can be separated by insulating materials, such as polymer matrix that does not have the particles 16 embedded therein or non-filled fibers/threads/yards. The creation of these distinct zinc cells create the micro and macroscopic charged particles in higher concentration in some places than in others and in some places, no charge at all. A conductive additive or conductive fiber may be used to connect the particle reservoirs 16 to behave electrically as a much larger cell. Metal concentrations may be controlled in a master batch before it is drawn as a bicomponent, the density of the filament, the ratio of filled/unfilled filaments making up the fiber/thread or yarn and ultimately the density of the fiber/thread/yard in the fabric. In this way more zinc may be recruited from the entire interconnected fiber, so it draws electrons easily from a larger reservoir.

The concentration of metal such as zinc in the binder or in the extruded fibers that forms the lines or dots determines the amount of metal available for the “battery”. The concentration may be 30% of the surface area of the fabric; however, the concentration of zinc may range from about 1% to about 99%. A mixture of binder and zinc metal may be formed as a paste and applied by silk screening and a 30% by weight zinc-to-binder is preferred for this. The line or dot width and length also determines the amount of metal in the deposition since the wider and longer the line, the more metal is available. Preferred line dots width is 1 mm width but width can vary from 0.1 mm up to 5 mm width. Since the deposition is on a fabric or carried in a substrate, the amount of binder/metal applied also can be varied. In certain embodiments, the fabric being coated can be coated twice or more times over the same pattern whereupon the thickness of the deposition can be increased as desired. In certain embodiments, the metal deposition area patterns cover from about 10% to about 90% of the surface area of the fabric. In other embodiments, the metal deposition areas cover from about 20% to about 80%, from about 15% to about 75%, from about 25% to about 50%, or from about 30% to about 40% of the surface area of the fabric or anywhere in between.

Although FIG. 1 shows the plurality of metal deposition areas 18 substantially uniformly distributed on the surface of the fabric, in other embodiments, the plurality of metal deposition areas may be randomly distributed on the surface of the fabric. Typically, the lines have a thickness of 0.1 to 3 mm, preferably 0.2 to 2 mm, more preferably 0.3 to 1.0, most preferably 0.4 to 0.5 mm. The spaced lines may be continuous and may take various forms including straight, curved and various angular shapes as shown, for example, straight continuous lines; straight broken lines; continuous saw-shaped; continuous wavy lines; broken wavy lines, etc, as described in our aforesaid '761 and '172 patents and our '901 patent and the lines may be approximately equal in thickness and are evenly spaced.

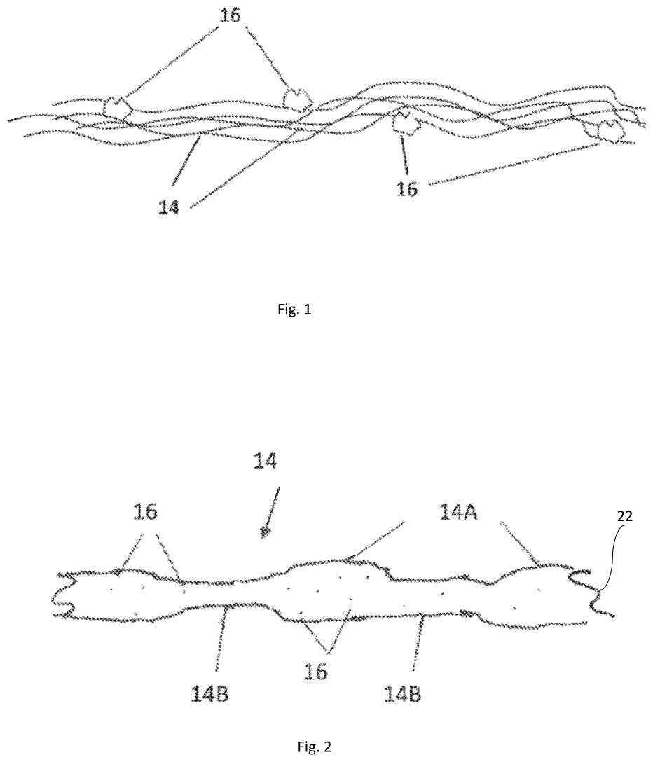

Referring to FIGS. 2 and 2A, a self-contained external battery-free iontophoresis apparatus in accordance with the present invention is formed following the teachings of our aforesaid '901 patent as follows: Metal particles, specifically elemental zinc particles, previously formed by grinding or precipitated out of suspension, and having an average particle size between 1 and 100 nanometers, more preferably 1-10 microns, even more preferably about 5 microns are mixed with a thermal plastic material such as polyethylene in a heated mixing vat 30 to melt the material, and the mixture extruded or melt spun at spinning station 32 to form fibers 34, having metal particles 36 contained therein. Polyethylene is the polymer of choice for releasing electrons from the metal. The porosity of the fiber may also be adjusted to affect the electrical field properties of the device 10. The metal-containing fibers may then be cabled or twisted at a cabling station 38 and woven at a weaving or knitting station 40 into a sheet or cloth. The resulting metal particle impregnated sheet or cloth is cut to size at a cutting station 42, assembled together with a substance to be absorbed by the user at an assembly station 44, and the assembled package formed into a self-contained battery-free iontophoresis apparatus at a laminating station 46. Lamination may be by application of glue on the border of base 12 and a metal-containing substrate 16, or by melt fusing the edges of the assembly.

The substrate 16 may also be impregnated with a compound that may be absorbed by the skin or favorably alter the appearance of the skin. The substrate may include ointment or cream reservoirs 20 to provide patches that can be applied to the user for amplified effect.

The substrate may alternatively or additionally be a polymer with a thickness that degrades over time due to solubility of the polymer or how the polymer is affected by the increased heat of being applied to skin. As the polymer degrades, new metal particles 16 suspended in the substrate may come into contact with the skin renewing the available metal to be oxidized. The substrate may be porous allowing it to form treatment reservoirs 20 to carry an amount of the substance to be absorbed. The amount of substance included in the reservoirs 20 may be calibrated to optimize absorption of the substance in the presence of electric fields created by the metal particles of the device.

Metal particles disposed on at least a portion of a surface of the substrate form a special pattern that positions the metal particles in discrete electrically isolated locations separated by a distance, wherein the fabric or substrate 14 is configured such that the metal particles contact a skin surface of the human or animal to form a plurality of half-cells of an air-metal battery, which alters skin membrane permeability and drives the substance into the skin.

In one aspect, the pattern of metal particles conform to blood circulation patterns and/or nerve or underlying muscle patterns of a human or animal. The metal particles comprise a metal, metal oxide or metal salt, preferably elemental zinc particles, zinc oxide particles, and zinc salt particles, or aluminum, iron, copper and magnesium, and an oxide or salt thereof. The device 10 may include an adhesive or adhesive tabs to fix the device to the skin of the animal or human. In the case where the device is fixed to the skin by an adhesive, the metal particles may be incorporated directly into the adhesive. Desired areas of application may include the face, below the eyes, the forehead, on a bald spot, at inflamed or infected areas, around a wound, near a blemish, mole, or discoloration, skin wrinkles, or other areas known to those having skill in the art.

In another embodiment, the device may be a mask or applicator ergonomically shaped to fit on a desired area of the skin. The device may have the substance to be absorbed impregnated into the substrate or may be placed over the substance that has already been applied to the skin. In another embodiment, the device 10 may be applied to the user and then the substance may be applied to the device 10.

The electric fields created by interaction of the device with the skin can increase absorption of a substance through the skin of a human or animal through providing a substance to contact the skin while also contacting a substrate having metal particles disposed on at least a portion of a surface of the substrate as a plurality of lines or dots in a spatial pattern that positions the metal particles in discrete electrically isolated locations separated by a distance.

The substance may be formulated to affect the electric field or fields. For example the substance may include an electrolyte to increase the interaction between the device and the skin. The battery of the device may be recharged through interactions of the metal particles with other ions that are available at the surface of the skin. In a particular embodiment, the electric field created by the iontophoretic battery is configured to change the permeability of the skin tissue. A field gradient may be provided that allows a medication or other compound to migrate through the cells that are subject to the electric field. The electric field may also be configured to improve biological absorption of natural products. For example, lactic acid byproduct from metabolism of muscles during strenuous activity leaves an acidic environment. In the presence of an electric field, permeability of the cell membranes changes and allows the removal of the lactic acid build up more quickly (recovery is faster).

Various changes may be made in the above invention without departing from the spirit and scope thereof. For example, the amount of zinc in the fabric or adhesive coating can be increased or decreased to change the battery capacity. Adding too much zinc to a fiber may weaken the fiber, so a bi-component concept may be used where only the outside layer has the metal and the inner core is polyester or another polymer that has more strength.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. The terms “including” and “such as” are not limiting and should be interpreted as “including, but not limited to,” and “such as, for example,” respectively. Moreover, any combination of the above-described embodiments in all possible variations thereof is encompassed by the disclosure unless otherwise indicated herein or otherwise clearly contradicted by context.

I claim:

1. A device for increasing absorption of substances through the skin of an animal or human, comprising:
a substrate;
a fixed desired pattern of particles of an oxidizable metal dispersed within the substrate in discrete electrically isolated locations separated by a distance, and exposed at least in part on a surface of the substrate; and
a treatment substance carried by the substrate,
wherein the metal particles form a plurality of half-cells of an air-or oxygen-metal battery, for ion exchange with the skin of the human or animal whereupon the treatment substance may be driven into the skin by iontophoresis.

2. The device of claim 1, wherein the pattern conforms to blood circulation patterns and/or nerve or underlying muscle patterns of the human or animal.

3. The device of claim 1, wherein the particles are 0.2 to 0.5 mm.

4. The device of claim 1, wherein the pattern covers from about 25% to 50%

5. The device of claim 1, wherein the metal particles comprise a metal, metal oxide or metal salt.

6. The device of claim 1, wherein the metal particles are selected from the group consisting of elemental zinc particles, zinc oxide particles, and zinc salt particles.

7. The device of claim 1, wherein the metal particles are metal particles selected from the group consisting of aluminum, iron, copper and magnesium, and an oxide or salt thereof.

8. The device of claim 1, further including a strap, an adhesive pad, or adhesive tabs adapted to fix the substrate to the user.

9. The device of claim 1, wherein the particles are substantially evenly spaced from one another.

10. The device of claim 9, wherein the particles are substantially evenly spaced at spacings from 0.2 to 0.5 mm.

11. A method for increasing absorption of a substance through the skin of a human or animal, comprising:
providing a device as claimed in claim 1, and disposing the device in contact with the skin, of a human or animal, whereupon the metal particles form a first half cell of a battery circuit, while the body of the human or animal forms a second half cell of the battery circuit with oxygen, whereupon the substance is driven into the skin of the human or animal by iontophoresis.

12. The method of claim 11, wherein the pattern conforms to blood circulation patterns and/or nerve or underlying muscle patterns of the human or animal.

13. The method of claim 11, wherein the particles are evenly spaced at spacings from 0.2 to 0.5 mm

14. The method of claim 11, wherein the particles are 0.2 mm to 0.5 mm wide.

15. The method of claim 11, wherein the pattern covers from about 25% to 50% of the surface of the cover layer.

16. The method of claim 11, wherein the metal particles comprise a metal, metal oxide or metal salt.

17. The method of claim 11, wherein the metal particles are selected from the group consisting of elemental zinc particles, zinc oxide particles, and zinc salt particles.

18. The method of claim 11, wherein the metal particles are metal particles selected from the group consisting of aluminum, iron, copper and magnesium, and an oxide or salt thereof.

19. The method of claim 11, further including fixing the device to the skin of the animal or human using adhesive.
